(19) United States
(12) Patent Application Publication
(10) Pub. No.: US 2002/0108619 A1
(43) Pub. Date: Aug. 15, 2002

(54) Method of preventing premature ejaculation

(76) Inventor: Hani A. Bouidani, Rowland Heights, CA (US)

Correspondence Address: Richard W. Goldstein, 2071 Clove Road, Staten Island, NY, 10304, US

(21) Appl. No.: 09/781,070

(22) Filed: Feb. 9, 2001

(51) Int. Cl. A61B 19/00

(52) U.S. Cl. 128/898

(57) ABSTRACT

A method of preventing premature ejaculation including the following steps: provide a natural stone; place the natural stone in a hand of a user; apply a quantity of water to the natural stone to create a wet natural stone; rub the wet natural stone on a penis of the user; wait about ½ hour; and perform sexual intercourse.

### BACKGROUND OF THE INVENTION

The present invention relates to method of preventing premature ejaculation and more particularly pertains to prolonging sexual intercourse.

The use of medical devices is known in the prior art. More specifically, medical devices heretofore devised and utilized for the purpose of controlling sexual emissions are known to consist basically of familiar, expected and obvious structural configurations, notwithstanding the myriad of designs encompassed by the crowded prior art which have been developed for the fulfillment of countless objectives and requirements.

By way of example, U.S. Pat. No. 5,063,915 to Wyckoff discloses a male genital device for controlling and delaying sexual emissions. U.S. Pat. Nos. 5,468,212 to Shooter and 5,535,758 to Hagihara disclose additional means for controlling sexual emissions.

While these devices fulfill their respective, particular objective and requirements, the aforementioned patents do not describe a method of preventing premature ejaculation for prolonging sexual intercourse.

In this respect, the method of preventing premature ejaculation according to the present invention substantially departs from the conventional concepts and designs of the prior art, and in doing so provides an apparatus primarily developed for the purpose of prolonging sexual intercourse.

Therefore, it can be appreciated that there exists a continuing need for a new and improved method of preventing premature ejaculation which can be used for prolonging sexual intercourse. In this regard, the present invention substantially fulfills this need.

### SUMMARY OF THE INVENTION

In the view of the foregoing disadvantages inherent in the known types of medical devices now present in the prior art, the present invention provides an improved method of preventing premature ejaculation. As such, the general purpose of the present invention, which will be described subsequently in greater detail, is to provide a new and improved method of preventing premature ejaculation, which has all the advantages of the prior art and none of the disadvantages.

To attain this, the present invention essentially comprises the following steps: provide a natural stone; place the natural stone in a hand of a user; apply a quantity of water to the natural stone to create a wet natural stone; rub the wet natural stone on a penis of the user; wait about ½ hour; and perform sexual intercourse.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved method of preventing premature ejaculation, which has all the advantages of the prior art medical devices and none of the disadvantages.

It is another object of the present invention to provide a new and improved method of preventing premature ejaculation, which may be easily and efficiently manufactured and marketed.

It is a further object of the present invention to provide a new and improved method of preventing premature ejaculation, which is of durable and reliable construction.

An even further object of the present invention is to provide a new and improved method of preventing premature ejaculation which is susceptible of a low cost of manufacture with regard to both materials and labor, and which accordingly is then susceptible of low prices of sale to the consuming public, thereby making such method of preventing premature ejaculation economically available to the buying public.

Even still another object of the present invention is to provide a new and improved method of preventing premature ejaculation for prolonging sexual intercourse.

Lastly, it is an object of the present invention to provide a new and improved method of preventing premature ejaculation including the following steps: provide a natural stone; place the natural stone in a hand of a user; apply a quantity of water to the natural stone to create a wet natural stone; rub the wet natural stone on a penis of the user; wait about ½ hour; and perform sexual intercourse.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a flow-chart view of the preferred embodiment of the method of preventing premature ejaculation constructed in accordance with the principles of the present invention.

### DESCRIPTION OF THE PREFERRED EMBODIMENT

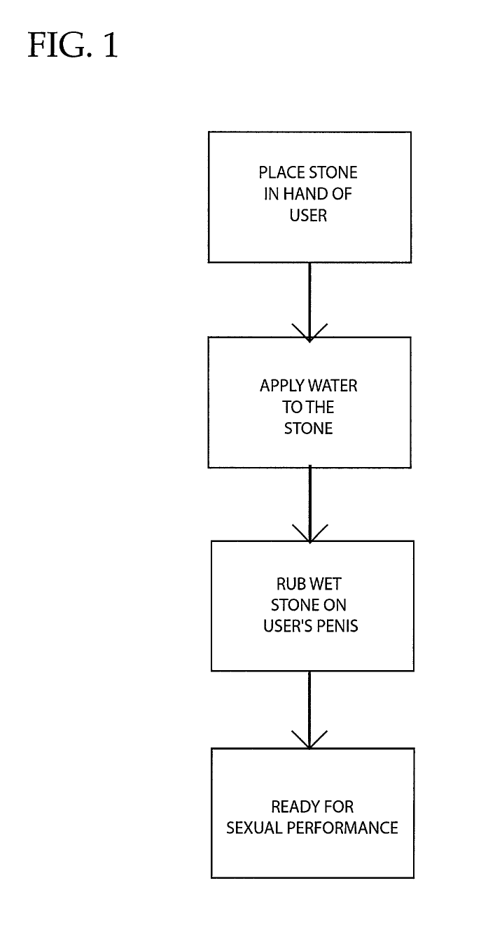

With reference now to the drawings, and in particular, to FIG. 1 thereof, the preferred embodiment of the new and improved method of preventing premature ejaculation embodying the principles and concepts of the present invention and generally designated by the reference number 10 will be described.

Specifically, it will be noted in the various Figures that the device relates to a method of preventing premature ejaculation for prolonging sexual intercourse.

The method of preventing premature ejaculation includes the following steps: provide a natural stone; place the natural stone in a hand of a user; apply a quantity of water to the natural stone to create a wet natural stone; rub the wet natural stone on a penis of the user; wait about ½ hour; and perform sexual intercourse.

The preferred stone used is a type of natural stone, preferably shale or similar type of rock. Additionally, the natural stone and the water can be combined to make a liquid formula that can be used directly on the penis.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and the manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modification and changes will readily occur to those skilled in the art, it is not desired to limit the invention to the exact construction and operation shown and described, and accordingly, all suitable modification and equivalents may be resorted to, falling within the scope of the invention.

What is claimed as being new and desired to be protected by Letters Patent of the United States is as follows:

1. A method of preventing premature ejaculation for prolonging sexual intercourse comprising, in combination:
providing a natural stone;
placing the natural stone in a hand of a user;
applying a quantity of water to the natural stone to create a wet natural stone;
rubbing the wet natural stone on a penis of the user;
waiting about ½ hour; and
performing sexual intercourse.
